(19) United States
(12) Patent Application Publication
(10) Pub. No.: US 2004/0224328 A1
(43) Pub. Date: Nov. 11, 2004

(54) siRNA screening method

(76) Inventors: Hans Prydz, Oslo (NO); Mohammed Amarzguioui, Oslo (NO); Torgeir Holen, Oslo (NO)

Correspondence Address: CHRISTIAN D. ABEL, ONSAGERS AS, POSTBOKS 6963 ST. OLAVS PLASS, NORWAY, N-0130, NO

(21) Appl. No.: 10/754,588

(22) Filed: Jan. 12, 2004

Related U.S. Application Data
(60) Provisional application No. 60/440,017, filed on Jan. 15, 2003.

(51) Int. Cl. C12Q 1/68

(52) U.S. Cl. 435/6

(57) ABSTRACT

A method for utilizing siRNA for the detection of optimal siRNA targeting sites capable of affecting the level of a target nucleic acid, comprising the targeting of one or more sites on an mRNA with one or more siRNA molecules and observing the level of the target nucleic acid.

This application claims the benefit under 35 USC §119 of U.S. provisional application 60/440,017 filed 15 January, 2003, the contents of which are incorporated herein by reference.

### FIELD OF THE INVENTION

The present invention regards a novel method for identifying potent siRNA sequences that may be used to modify the expression of a target gene sequence. Further, the present invention provides siRNA molecules identified through the present screening method and pharmaceutical preparations comprising said siRNA molecules.

### BACKGROUND OF THE INVENTION

Antisense effects were in C. elegans found both with the antisense and, surprisingly, the sense strand of RNA (1), leading up to the discovery of RNA interference (RNAi) by Andrew Fire and co-workers (2). The potent RNAi process, whereby dsRNA causes specific interference with the expression of homologous endogenous genes, appears to be defence against virus and transposons. This defence has subsequently been shown to exist in a wide range of species (3-5). With the demonstration of the efficacy of short interfering RNAs (siRNA) in human cells (6-8), a valuable tool for both research and therapeutics was created. Now the development have come a full circle with the recent reports that the antisense strand of siRNA (RNAi antisense) is almost as potent as the siRNA duplex (9-11).

The mechanism of action of the RNA interference pathway is still not fully understood and different theories are proposed. Long dsRNA is first processed to shorter 21-23 nt fragments, i.e. siRNA, by an enzyme named Dicer. In the second step the siRNAs produced combine with, and serve as guides for, a ribonuclease complex called RNA-induced silencing complex (RISC), which cleaves the homologous single-stranded mRNAs. However, the siRNA appears to be incorporated into an inactive RISC complex, requiring unwinding of the duplex with concomitant loss of its sense strand for conversion into an active complex (RISC*) (12). RISC cuts the mRNA approximately in the middle of the region paired with the antisense siRNA, after which the mRNA is further degraded.

The RNAi antisense is also incorporated into RISC in HeLa cell extracts and supports RISC-specific target RNA cleavage although at lower efficiency than the siRNA duplex (9, 10). The highly diverging estimates reported for the size of RISC (10, 12, 13), together with the reports of additional RISC-like complexes (14-17) associated with both siRNA and the related microRNAs (18), suggest the existence of various distinct complexes with possible involvement in different RNA interference pathways.

Since the discovery of RNA interference, and despite of the indistinctness regarding the mechanism of action and the applicability of RNA interference, there has been a perceptible growing interest in identifying siRNA molecules and developing new drugs towards a range of conditions. WO 01/75164 regards RNA sequence-specific mediators of RNA interference and relates to isolated RNA molecules (double stranded; single stranded) of from about 21-23 nucleotides in general. WO 01/75164 also relates inter alia to a method of producing said RNA molecules, e.g. by using the Drosophila in vitro system, by chemical synthesis or recombinant techniques. Further, WO 02/44321 disclose inter alia isolated double-stranded RNA molecule of 19-25 nucleotides capable of target-specific nucleic acid modifications, a method for processing said RNA molecules and the use thereof.

Recently, great many researchers and pharmaceutical companies have seen the advantage of modulating gene expression by utilizing RNA interference. For example, WO 02/101072 claims methods for modulate the expression of LETM-1 (leucine zipper EF hand transmembrane receptor) by administering a siRNA to a subject. Similarly, WO 02/101072 discloses the modulation of a CD43 encoding nucleic acid by using e.g. siRNA molecules. WO 02/096927 and WO 02078610 disclose methods to affect the expression of vascular endothelial growth factor receptor (VEGF) or PAK2 respectively. Further, WO 02/085289 regards the development of medicaments for the modulation of angiogenesis and thereby identifying siRNA molecules that inhibit the expression of a nucleic acid encoding C1 angiogenesis protein (integrin-linked kinase associated protein, ILKAP)). However, WO 02/101072, WO 02/096927, WO 02078610 and WO 02/085289 do not disclose examples of specific siRNA candidates against the respective target genes.

Also, none of the previous mentioned publications takes into account the fact that even though all gene expression can, in principle, be suppressed by use of e.g. oligonucleotide (synthetic chains), ribozymes or siRNA molecules; there is no reliable way to determine exactly what part of an mRNA sequence is most effectively targeted by siRNA. The position dependence of siRNA efficacy is further supported in PCT/NO03/00045, hereby incorporated by reference, which demonstrates that without revealing any unusual features, siRNA sequences directed towards different sequences of the same mRNA had quite dissimilar efficiency.

This finding of position dependence is contrary to the currently held belief regarding RNA interference in the prior art. It is generally believed that the identification of an effective siRNA is troubled by only “occasional ineffectiveness” (21, 22). Also, Thomas Tuschl has previously claimed that RNA interference does not need to be selected for an “optimal” sequence (23). Further, Tuschl and colleagues have predicted “that it will be possible to design a pair of 21- or 22-nt RNAs to cleave a target RNA at almost any given position” (7).

Because our findings that specific regions of the mRNA are far more efficient in gene silencing than others, the necessity for, and method of finding optimal siRNA target sequences according to the present invention is clearly surprising in light of the prior art.

Traditionally, chemical modification of nucleic acids has inter alia been used to protect single stranded nucleic acid sequences against nuclease degradation and thus obtaining sequences with longer half life. For example, WO 91/15499 discloses 2′O-alkyl oligonucleotides useful as antisense probes. Also, 2-O-methylation has been used to stabilize hammerhead ribozymes (4). However, little is known about the effects of chemical modifications of siRNAs. Further, the presence of large substituents in the 2′hydroxyl of the 5′terminal nucleotide might interfere with the proper phosphorylation of the siRNA shown to be necessary for the activity of the siRNA (12).

Thus, an inherent differential activity in the various siRNAs in a population would mean that different siRNAs would be affected by siRNA modifications, chemical or mutational, in different ways, and generally in a deleterious way, as shown in the exemplary material. Therefore, there is a considerable need for a method to efficiently identify optimal siRNA molecules, which may or may not be chemically modified, to be able to develop useful pharmaceutical agents to modulate the expression of a target gene.

There have been proposals for full-genome screenings by utilizing the RNAi pathway in C. elegans to e.g. validate potential drug targets, investigate the biological role of validated targets and screen for active compounds (O'Neil et al., (24). Also, EP 0 756 634 B1 discloses a method for the screening of a genetic sequence which is capable of inhibiting, reducing, altering or otherwise modulating the expression of a target nucleotide sequence, e.g. the screening for useful antisense, sense or ribozyme constructs or other nucleic sequences. More specifically, the method disclosed in EP 0 756 634 B1 makes use of S. pombe to evaluate the effect of the introduction of the molecule to be tested on the expression of a target gene in S. pombe.

However, the prior art does not disclose a suitable method that may be used to distinguish between efficient and less efficient siRNA candidates that render it possible to develop new useful therapeutic approaches to a variety of disorders. The present invention provides a method for identifying potent siRNA molecules.

### SUMMARY OF THE INVENTION

The present invention provides a method of detecting optimal siRNA targeting sites by targeting a series of sites on an mRNA. The preparation of the siRNA molecules to be used and the determination of the optimal siRNA molecules according to the method of the present invention may be provided by various techniques as will be apparent from the description of the invention below. More specifically, in one embodiment, the method according to the present invention comprises the following steps:

a) providing a suitable range of siRNA molecules directed towards the target nucleic acid;

b) introducing each of the siRNA molecules into a cell or a system containing the target nucleic acid;

c) determining the effect of the siRNA molecules on the level of said target nucleic acid; and

d) identifying the optimal siRNA molecules from the effects determined in (c).

Preferably, the siRNA molecules are provided by e.g. chemical synthesis, in vitro transcription or by endogenous expression directed by a suitable DNA vector.

In one embodiment, the siRNA molecules are introduced into a suitable cell line, more preferably a human keratinocyte cell line HaCaT.

Further, according to one embodiment of the present invention, the siRNA molecules are introduced by cationic liposome transfection, more preferably by Lipofectamin™2000 transfection.

Also, according to other embodiments of the present invention, the siRNA molecules are introduced by e.g. electroporation, microinjection or by coated gold particles shot into cells (GeneGun in plant cells).

In another embodiment of the present invention, the siRNA molecules are introduced by expression from a suitable vector, wherein the vector is introduced according to any of the previous mentioned methods for introducing the SiRNA molecules into a cell.

In yet another embodiment of the present invention, the siRNA molecules are introduced into a suitable cell lysate.

In still another embodiment the siRNA molecules are introduced into a suitable in vitro expression assay.

Also, according to various embodiments of the present invention, the siRNA efficacy is determined e.g. by Northern blotting analysis, qRT-PCR, Western analysis, primer extension analysis. In still another embodiment of the present invention, the siRNA efficacy is determined by fluorescent marker.

Further, according to still another embodiment the siRNA efficacy is determined by phenotypic marker. More preferably, the phenotypic marker indicates a specific morphological, proliferative or apoptotic characteristics.

In another aspect, the present invention also provides the use of a method according to any of the following claims for the determination of optimal siRNA targeting sites.

Additional aspects will be set forth in part in the description, or may be learned by practice of the invention. It is to be understood, however, that both the foregoing general description and the following detailed description are exemplary and explanatory only and are not to be contemplated as restrictive to the scope of the present invention.

The accompanying drawings, which are incorporated in and constitute a part of this specification, illustrate embodiments of the inventions and thus explains the principles of the present invention.

### BRIEF DESCRIPTION OF THE FIGURES

FIG. 1 siRNAs, reporter construct and RNAi of transgene expression; a) The sense (top) and antisense (bottom) strands of siRNA species targeting eight sites within human TF (Genbank entry Acc. No. M16553) mRNA are shown, b) Luciferase reporter construct of human TF and c) RNAi by siRNA in cotransfection assays (averages of three or more independent experiments each in triplicate, ±s.d. are shown).

FIG. 2 Efficacy of the siRNAs in standard cotransfection assays in HaCaT cells. Different synthetic batches of the hTF167i siRNA showed similar efficacy. Results are averages of at least three experiments, each in triplicate.

FIG. 3 siRNA mediated reduction of endogenous TF expression; a) hTF167i and hTF372i induced cleavage of mRNA in transfected cells. The Northern analysis of TF mRNA was performed after transfection of HaCaT cells with siRNA (100 nM) with GADPH as control. Arrowhead indicates cleavage fragments resulting from siRNA action, b) Measurements of the effect of siRNAs on steady state mRNA levels (filled bars), procoagulant activity (dotted bars) and TF protein (antigen) expression (hatched bars) show that siRNA reduces mRNA, TF antigen levels and procoagulant activity. For measurement of procoagulant activity and antigen, cells were harvested 48 h after si transfection to accommodate the 7-8 h half-life of TF protein. Data are from a representative experiment in triplicate.

FIG. 4 Dose-response curve for hTF167i.

FIG. 5 Time-dependence of siRNA-mediated RNAi; a) Inhibitory activity is reduced when mutations (M1 and M2 refer to one and two mutations, respectively) are introduced into the siRNAs. Cells were transfected with 100 nM siRNA and harvested for mRNA isolation 4, 8, 24 and 48 h (filled bars, lined bars, white bars with black dots and hatched bars, respectively). Expression levels were normalised to GADPH and standardised to mock-transfected cells at all time-points, b) Time-course of decay of inhibitory effect for mRNA levels (closed diamonds), reporter gene activity (open triangles) and procoagulant activity (filled bars).

FIG. 6 siRNA modifications. (A) Mutated and wild type versions of the siRNA hTF167i. The sequence of the sense strand of wild type (wt) siRNA corresponds to position 167-187 in human Tissue Factor (Ass. No. M16553). Single (s1, s2, s3, s4, s7, s10, s11, s3, s16) and double mutants (ds7/10,ds10/11, ds10/13, ds10/16) are all named according to the position of the mutation, counted from the 5′end of the sense strand. All mutations (in bold) are GC inversions relative to the wild type. (B) Chemically modified versions of the siRNA hTF167i. Non-modified ribonucleotides are in lower case. Phosphorothioate linkages are indicated by asteriscs (*), while 2′-O-methylated and 2′-O-allylated ribonucleotides are in normal and underlined bold upper case, respectively.

FIG. 7 Activity of mutants against endogenous hTF mRNA. HaCaT cells were harvested for mRNA isolation 24 h post-transfection. TF expression was normalised to that of GAPDH. Normalised expression in mock-transfected cells was set as 100%. Data are averages+s.d. of at least three independent experiments.

FIG. 8 Activity of chemically modified siRNA against endogenous TF mRNA. Experiments were performed and analysed as described in FIG. 7.

FIG. 9 Persistence of TF silencing by chemically modified siRNAs. A) Specific TF expression 5 days post-transfection of 100 nM siRNA. B) Time-course of TF mRNA silencing. Cells harvested 1-3-5 days after single transfection of 100 nM siRNA. Medium was replaced every second day.

### DETAILED DESCRIPTION OF THE INVENTION

The present invention will now be described in more detail with reference to the examples. The examples serve only as a representative group of the various embodiments of the present invention. The list of examples is thus not meant to be exclusive.

The term “siRNA” or “siRNA molecule” as used herein means—definition: double stranded RNA molecules in which each strand comprises 19-29 nucleotides that may or may not be chemical modified.

The term “chemical modification of the siRNA (molecule)” or “chemical modified siRNA (molecule)” as used herein means any chemical modification of said RNA sequence. Non-limited examples of such chemical modifications are 2′-OH-modification, e.g. alkylation such as methylation; 3′ or 5′ end modifications such as fluorescent labels, non-standard nucleotides, lipophilic linker molecules or peptides; or modification or exchange of the phosphodiester bond, e.g. with phosporothioates, methylphosphonates, or polyamide.

The term “suitable range of siRNA” as used herein means any set of unique sequences or a mixture thereof, including a randomized collection of such molecules, provided by any of the means previously stated.

There is a commonly held belief that mRNA is generally accessible to siRNA. However, the results presented herein demonstrate the clear differences in activity of various siRNA molecules. Also, even if most siRNAs have some activity, some applications may require the identification of the best siRNA. This would be of particular importance for therapeutic applications requiring modifications for example increasing the in vivo stability of siRNA or for delivery of siRNA in vivo. Since such modifications gradually reduce activity (11), only the most effective siRNA would have the necessary excess capacity to tolerate modification and still retain sufficient activity for mRNA targeting.

According to another aspect of the invention, various steps in the method can automated. An automated search for good target positions would be significantly cheaper and simpler to perform In the case of chemically synthesized siRNAs, automation can be accomplished through streamlining of the following technologies:

1. automated RNA synthesis

2. automated transfection by robotic mixing of RNA, transfection agent and cells

3. automated isolation of total RNA or mRNA (ref QIAGEN)

4. automated determination of target mRNA expression by quantitative real-time RT-PCR (ref QIAGEN or Perkin Elmer)

Because the siRNA is double stranded, an automated method would require the annealing of the separately-synthesized sense and anti-sense strands. This step could be automated as well, for example, by providing robotic means to pipette the sense and anti-sense RNA from separate solutions into a new receptacle, wherein annealing occurs through controlled heating and gradual cooling under conditions that prevent evaporation.

The screening strategy can also be performed with in vitro transcribed RNA (resulting in sense and anti-sense strands that afterward are annealed as described above), starting with the synthesis of a DNA strand consisting of the complementary strand of the particular strand of the siRNA in conjunction with the sequence of the minus strand of a phage (T7, T3, Sp6) RNA polymerase promoter. Annealing of the above DNA strand with the sense strand of the promoter would yield a template for in vitro transcription of RNA. Following in vitro transcription (and preferably a purification step), transfection and further analysis would proceed as described for chemically synthesized RNA.

A mixture of siRNA can be obtained by enzymatic digestion (by either Dicer or RNaseIII) of in vitro transcribed longer dsRNA. In this case, following transfection with the RNA mixture and total RNA isolation, a different analysis step would be required to detect the most effective siRNA molecules. The preferable way to do this is by primer extension analysis, which identifies the most prominent cleavage positions within the target mRNA. From the known sequence of target mRNA, the sequence of siRNA causing the most prominent cleavage events can be inferred.

### EXAMPLES

The invention will now be described by way of examples. Although the examples represent preferred embodiments of the present invention (best mode), they are not to be contemplated as restrictive or limiting to the scope of the present invention and the enclosed claims.

Materials and Methods

siRNA Preparation

21-nucleotide RNAs according to SEQ ID's 1-41 were chemically synthesized using phosphoramidites (Pharmacia and ABI). Deprotected and desilylated synthetic oligoribonucleotides were purified by reverse phase HPLC. Ribonucleotides were annealed at 10 μM in 500 μl 10 mM Tris-HCl pH 7.5 by boiling and gradual cooling in a water bath. Successful annealing was confirmed by non-denaturing polyacrylamide gel electrophoresis. siRNA species were designed targeting sites within human Protein serine kinase HI (PSKH1) (Acc.No. AJ272212) and human Tissue Factor (TF) (Acc.No. M16553) mRNAs.

Cell Culture

HeLa, Cos-1 and 293 cells were maintained in Dulbecco's Minimal Essential Medium (DMEM) supplemented with 10% foetal calf serum (Gibco BRL). The human keratinocyte cell line HaCaT was cultured in serum-free keratinocyte medium (Gibco BRL) supplemented with 2.5 ng/ml epidermal growth factor and 25 μg/ml bovine pituitary extract. All cell lines were regularly passaged at sub-confluence and plated one or two days before transfection. Lipofectamine-mediated transient co-transfections were performed in triplicate in 12-well plates with 0.40 μg/ml plasmid (0.38 μg/ml reporter and 20 ng/ml control) and typically 30 nM siRNA (0.43 μg/ml) essentially as described (29). Luciferase acitivity levels were measured in 25 μl cell lysate 24 h after transfection using the Dual Luciferase assay (Promega). Serial transfections were performed by transfecting initially with 100 nM siRNA, followed by transfection with reporter and internal control plasmids 24 h before harvest time points. For Northern analyses and coagulation assays, HaCaT cells in 6-well plates were transfected with 100 nM siRNA in serum-free medium. For endogenous targets, Lipofectamine2000™ was used for higher transfection efficiency. Poly(A) mRNA was isolated 24 h after transfection using Dynabeads oligo(dT)₂₅ (Dynal). Isolated mRNA was fractionated for 16-18 h on 1.3% agarose/formaldehyde (0.8 M) gels and blotted on to nylon membranes (MagnaCharge, Micron Separations Inc.). Membranes were hybridised with random-primed TF (position 61-1217 in cDNA) and GAPDH (1.2 kb) cDNA probes in PerfectHyb hybridisation buffer (Sigma) as recommended by the manufacturer.

TF Activity and Antigen

For TF activity measurements HaCaT cell monolayers were washed thrice with icecold barbital buffered saline pH 7.4 (BBS, 3 mM sodium barbital, 140 mM NaCl) and scraped into BBS. Immediately after harvesting and homogenisation the activity was measured in a one-stage clotting assay using normal citrated platelet poor plasma mixed from two donors and 10 mM CaCl₂. The activity was related to a standard (29,30). One unit (U) TF corresponds to 1.5 ng TF as determined in the TF ELISA (30,31). The activity was normalised to the protein content in the cell homogenates, as measured by the BioRad Bradford or DC assays. TF antigen was quantified using the Imubind Tissue Factor ELISA kit (American Diagnostica, Greenwich, Conn., USA). The samples were left to thaw at 37° C. and homogenised. An aliquot of each homogenate (100 μl) was diluted in phosphate-buffered saline containing 1% BSA and 0.1% Triton X-100. This sample was then added to the ELISA-well and the procedure from the manufacturer followed. The antigen levels were normalised to the total protein content in the cell homogenates.

### Example 1

### Analysis of hTF siRNA Efficacy in Cells Transiently Cotransfected with hTF-LUC and hTF siRNA (i.e. Analysis of RNAI by siRNA(s) in Cotransfection Assays)

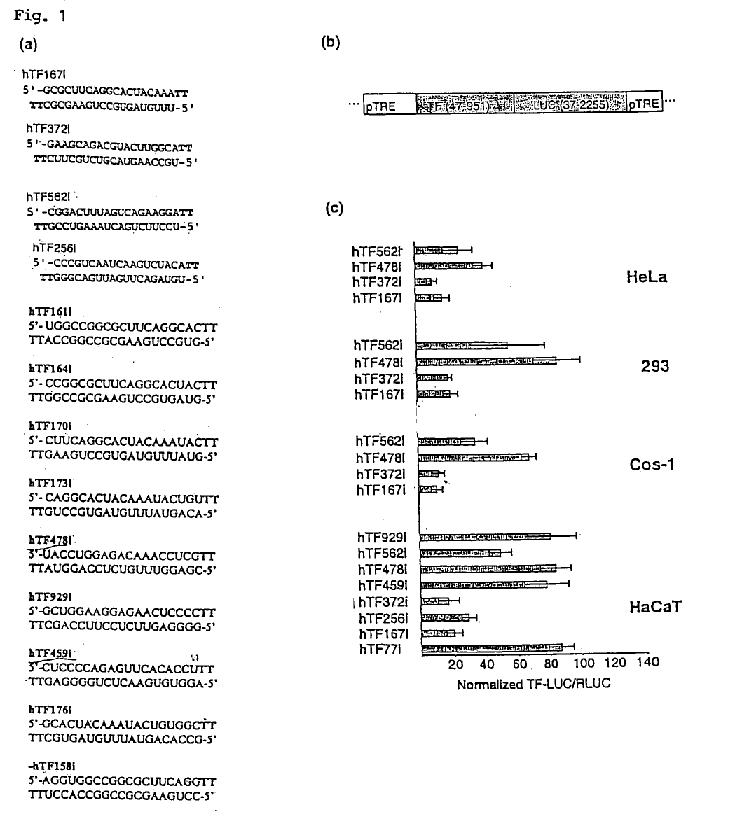

The initial analysis of TF siRNA efficacy was performed in HeLa cells transiently cotransfected with hTF-LUC (FIG. 1b) and hTF siRNA (FIG. 1a) using the Dual Luciferase system (Promega). Ratios of LUC to Rluc expression were normalised to levels in cells transfected with a representative irrelevant siRNA, Protein Serine Kinase 314i (PSK314i).

The siRNAs had potent and specific effects in the cotransfection assays, with the best candidates, hTF167i and hTF372i, resulting in only 10-15% residual luciferase activity in HeLa cells (FIG. 1c). Furthermore, also a positional effect was found, as hTF562i showed only intermediate effect, and hTF478i had very low activity. This pattern was also found in 293, COS-1 and HaCaT cells (FIG. 1c), and with siRNAs from different synthetic batches and at various concentrations (the siRNAs caused the same degree of inhibition over a concentration range of 1-100 nM in cotransfection assays; data not shown).

Coculturing siRNA transfected cells with reporter plasmid transfected cells, both in HeLa cells and in the contact-inhibited growth of HaCaT cells, gave no indication of siRNA transfer between cells (data not shown), despite the medium-mediated transfer previously reported by other investigators (25).

### Example 2

### Investigation of siRNA Position-Dependence at Codon-Level Resolution

The accessibility of the region surrounding the target site of the best siRNA (i.e. hTF167i) at a higher resolution was investigated. siRNAs (hTF158i, hTF161i, hTF164i, hTF170i, hTF173i and hTF176i) were synthesized which targeted sites shifted at both sides of hTF 167i in increments of 3 nts, wherein each of them shared 18 out of 21 nts with its neighbours (see FIG. 1c). Surprisingly it was found that despite the minimal sequence and position-differences between these siRNAs, they displayed a wide range of activities (FIG. 2). There was a gradual change away from the full activity of hTF167i that was more pronounced for the upstream siRNAs. The two siRNAs hTF158i and hTF161i were shifted only nine and six nucleotides away, respectively, from hTF167i, yet their activity was severely diminished. These results suggest that local factor(s) caused the positional effect.

### Example 3

### Analysis of hTF siRNA Efficacy on Endogenous mRNA

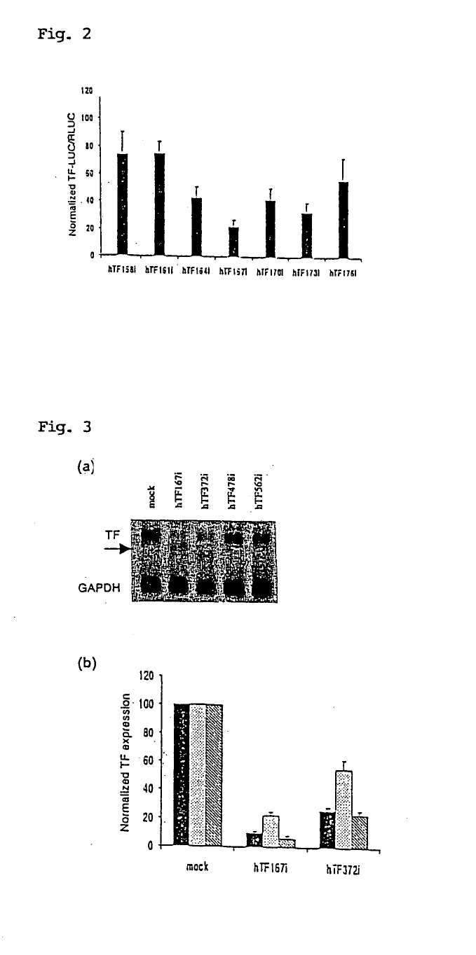

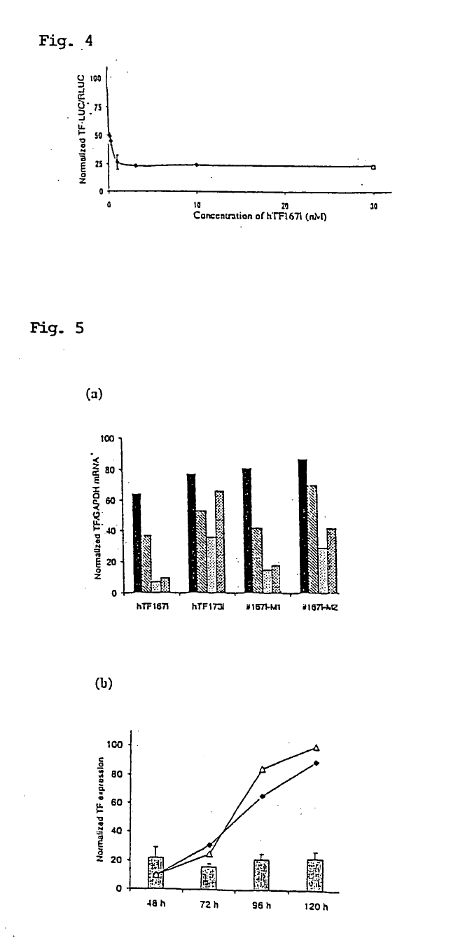

The results of cotransfection assays involving the use of forced expression of reporter genes as substrates may be difficult to interpret. The effect of siRNA was therefore also measured on endogenous mRNA targets in HaCaT cells (FIG. 3a) which express TF constitutively. The two best TF siRNAs, hTF167i and hTF372i, showed strong activity also in this assay, as normalised TF mRNA was reduced to 10% and 26%, respectively (FIG. 3a). Interestingly, cleavage products, whose sizes were consistent with primary cleavages at the target sequences, were clearly visible below the depleted main band, though cleavage assays of mRNA based on RNAi have so far failed in mammalian systems (26). Thus, the present invention also relates to siRNA which is able to cleave mRNA in mammalian cells. Furthermore, the observed effect suggests that RISC may be active also in mammals. The third best siRNA in cotransfection assays, hTF256i, also resulted in significant depletion of TF mRNA levels (57% residual expression, data not shown). The remaining TF siRNA did not show any activity as measured by Northern assays (FIG. 3b), nor did they stimulate TF expression, a point of some interest, as transfection with chemically modified ribozymes can induce TF mRNA three-fold (data not shown). Thus, this relative inertness of irrelevant siRNAs (i.e. siRNAs with <<non-specific>>effects) further enhances the promise of siRNA-based drugs.

The coagulation activity in the HaCaT cells was reduced 5-fold and 2-fold, respectively, in cells transfected with hTF167i and hTF372i, compared to mock-transfected cells (FIG. 3b and FIG. 5b). The effect of siRNAs on total cellular TF protein was also measured (FIG. 3b), and demonstrated an inhibitory effect that was generally greater than the observed effect on procoagulation activity. Thus, according to the present invention, we conclude that the siRNAs hTF167i and hTF372i display specificity and potency in a complex physiological system, and that we have demonstrated positional effects, as other siRNA molecules against the same target mRNA are basically inactive. Data from a new series of TF siRNA are in support of this conclusion (data not shown), and this inactivity of certain siRNAs might be due to mRNA folding structure or blockage of cleavage sites by impenetrable protein coverage (6).

### Example 4

### Analysis of the Time-Course and Persistence of siRNA Silencing

The time-course of mRNA silencing was measured, and Northern analysis of cells harvested 4, 8, 24 and 48 hours after start of transfection showed maximum siRNA silencing after 24 hours (FIG. 5a). There seemed to be a difference in the apparent depletion rate, as hTF167i reduced the mRNA level more than hTF173i at each time-point. Similar observations were made for modified versions of hTF 167i, in which the induced mutations (M1 and M2) resulted in reduced inhibitory activity. Mutations in the anti-sense strand had a more pronounced effect than the corresponding mutations in the sense strand. The fact that siRNA-induced target degradation was incomplete (a level of approximately 10% of the target mRNA remained even with the most effective siRNAs), may be due to the presence of a fraction of mRNA in a protected compartment, e.g. in spliceosomes or in other nuclear locations. However, in view of the above data and data from competition experiments, a more likely possibility may be a kinetically determined balance between transcription and degradation, the latter being a time-consuming process.

Experiments in plants (27) and nematodes (12,13) have suggested the existence of a system whereby certain siRNA genes are involved in the heritability of induced phenotypes. To investigate the existence of such propagators in mammalian cell lines, the persistence of the siRNA silencing in HaCaT cells transfected at a very low cell density was measured. In an experiment based on serial transfection of reporter constructs there was a gradual recovery of expression between 3 and 5 days post-transfection, and the time-dependence of the siRNA effect on endogenous TF mRNA was similar (FIG. 5b). The level of TF mRNA in mock-transfected control cells declined gradually during the experiment, in what appeared to be cell expansion-dependent down-regulation of expression. Interestingly, the procoagulant activity showed little indication of recovering to control levels in transfected cells (FIG. 5b, columns). Similar observations were made with hTF372i and with a combination of hTF167i, hTF372i and hTF562i (data not shown).

### Example 5

### Analysis of the Effect of Introducing Base-Pairing Mutations in the siRNA Sequences

As mentioned previously, the present siRNA were mapped more systematically in order to determine whether mutations were equally tolerated within the whole siRNA. A total of 8 different new single-mutant siRNA were designed and named according to the position (starting from the 5′ of the sense strand) of the mutation (s1, s2, s3, s4, s7, s11, s13, s16, i.e. SEQ ID NO 9-17). The previously described central single-mutant M1 (eksempel 4) was included in this analysis and renamed s10. All siRNAs were analysed for productive annealing by denaturing PAGE (15%).

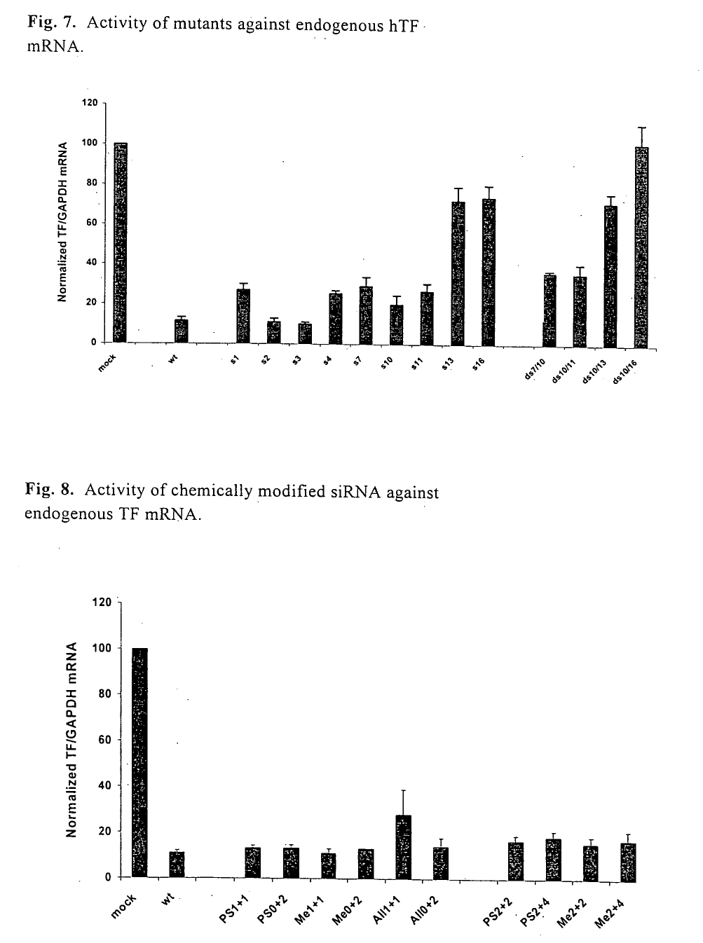

All the various mutant siRNAs were analysed for depletion of endogenous TF mRNA in HaCaT cells, 24 h after LIPOFECTAMINE2000-mediated transfection, as previously described. A summary of the data is shown in FIG. 7. The wild type siRNA, and the mutant s10, included as positive controls, depleted TF mRNA to ca 10% and 20% residual levels, as expected and previously reported. The activities of the other mutants fall in three different groups depending on their position along the siRNA. Mutations in the extreme 5′ end of the siRNA (s1-s3) were very well tolerated, exhibiting essentially the same activity as the wild type. Mutations located further in, up to the approximate midpoint of the siRNA (s4, s7, s10, s11), were slightly impaired in their activity, resulting in depletion of mRNA to 25-30% residual levels. Both the mutations in the 3′ half of the siRNA, however, exhibited severely impaired activity. This suggested to us a bias in the tolerance for mutations in the siRNA. The activities of several double mutants, in which the central position (s10) was mutated in conjunction with one additional position (s7, s11, s13, s16), were also analysed. The bias in mutation tolerance was also evident for these double mutants, as the rank order of their activity mirrored that of the activity of the single mutants of the variant position. This observation strengthens the proposition that the differential activity of mutants is due to an intrinsic bias in the tolerance for target mismatches along the sequence of the siRNA. The reason for such a bias might be linked to the proposed existence of a ruler region in the siRNA which is primarily used by the RISC complex to “measure up” the target mRNA for cleavage (28).

### Example 6

### Effects of Chemical Modification of the siRNA Sequences

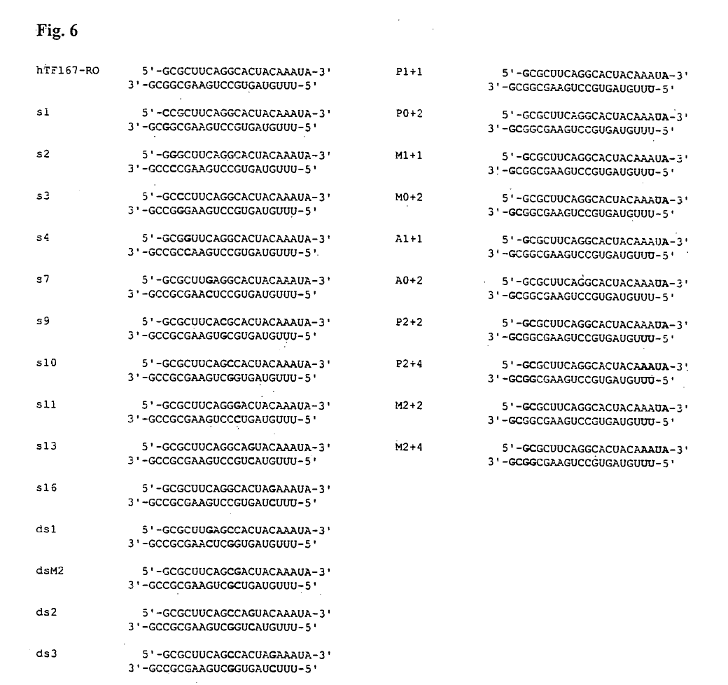

A series of siRNAs with one modification each in the extreme 5′ and 3′ ends of the siRNA strands [P1+1, M1+1, A1+1, i.e. SEQ ID NO 22(32), 26(36) and 30(40), respectively (the numbers in parentheses represent the SEQ ID of the complementary second strand)] was initially synthesized. The 5′ end of the chemically synthesized siRNAs might be more sensitive to modification since it has to be phosphorylated in vivo to be active (12). We therefore also included siRNAs with two modifications only in the non-base pairing 3′ overhangs (siRNAs P0+2, M0+2 and A0+2, i.e. SEQ ID NO 23(33), 27(37) and 31(41), respectively, cf. FIG. 6), which were known to be tolerant for various types of modifications (33, 32, 7). Northern analysis of transfected HaCaT cells demonstrated essentially undiminished activity of all the modified siRNAs, with the exception of the siRNA with allylation at both ends (FIG. 8). Allyl-modification in the 3′ end only had no effect on activity. The presence of a large substituent in 2′-hydroxyl of 5′ terminal nucleotide might interfere with the proper phosphorylation of the siRNA shown to be necessary by Nykanen et al (12).

We next wanted to know if any of these mutations were sufficient to increase the persistence of siRNA-mediated silencing. Endogenous TF mRNA recovers gradually 3-5 days after transfection with wild type siRNA targeting hTF167. Transfecting HaCaT cells with active and chemically modified siRNA in parallel, we could not demonstrate any significant difference in the silencing activities 3 and 5 days post-transfection (data not shown). The moderate modifications we had introduced, although exhibiting full initial activity, were therefore clearly not sufficient to substantially stabilize the siRNAs in vivo.

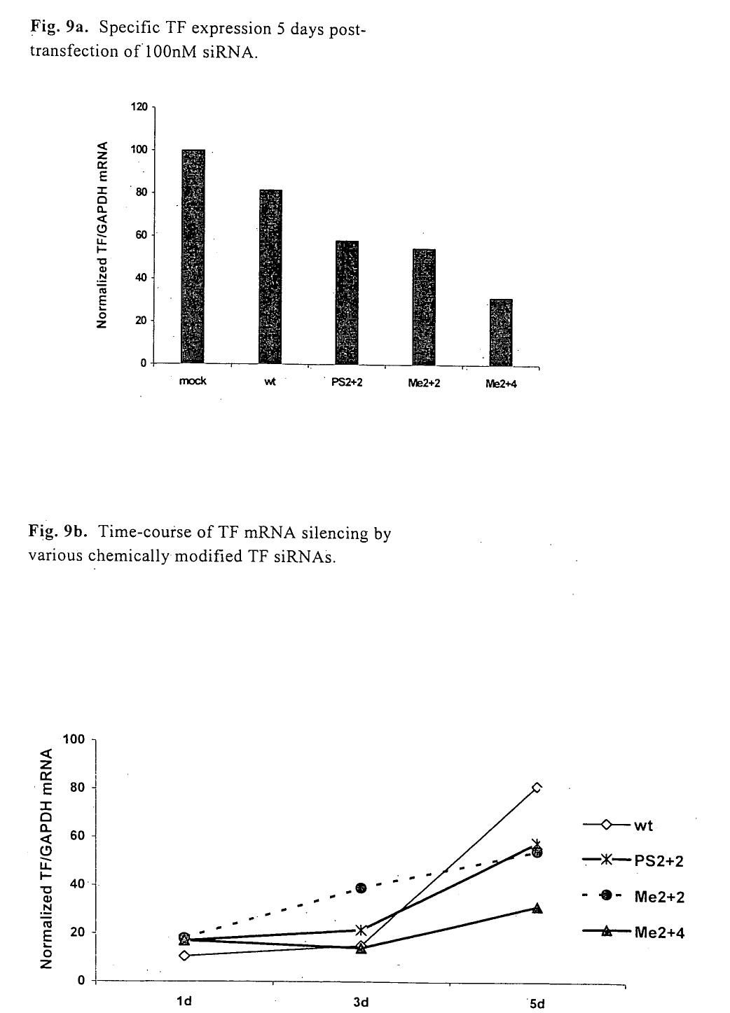

With the activity of the siRNA still intact after our initial moderate modifications, the degree of modifications was extended to include either two on both sides or two on the 5′ end in combination with four in the 3′ end. Due to the less promising results with the allylated versions from the first series, and the higher cost associated with these modifications, we decided to restrict ourselves to phophorothioate modifications and methylations for the next series. The new set of siRNAs were likewise analysed for initial activity 24 h following transfection into HaCaT cells. Normalized expression levels in cells transfected with modified siRNAs were slightly elevated, at 16-18% residual levels compared to 11% in cells transfected with wild type. The most extensively phosphorothioated siRNA proved to be toxic to cells, resulting in approximately 70% cell death compared to mock-transfected cells (measured as the expression level of the control mRNA GAPDH). Due to these complications, this siRNA species was not included in further analysis. The remaining siRNA species were evaluated for increased persistence of silencing by analysing TF mRNA expression 5 days after a single transfection of 100 nM siRNA. At this point, TF expression in cells transfected with wild type siRNA had recovered almost completely (80% residual expression compared to mock-transfected cells) (FIG. 9a). In cells transfected with the most extensively modified siRNA (M2+4; SEQ ID NO 29(39)), however, strong silencing was still evident (32% residual expression). The less extensively modified siRNA species (P2+2, M2+2; SEQ ID NO 24(34) and SEQ ID NO 28(38) respectively), although less effective than Me2+4, consistently resulted in lower TF expression 5 days post-transfection (55-60%) than the wild type. Time-course experiments demonstrated that the wild type siRNA was still the most effective 3 days post-transfection, when silencing was relatively unimpaired, but that silencing drops off at a much higher rate thereafter (FIG. 9b).

### REFERENCES

1. Guo, S. and Kemphues, K. (1995) par-1, a gene required for establishing polarity in C. elegans embryos, encodes a putative Ser/Thr kinase that is asymmetrically distributed. Cell, 81, 611-620.

2. Fire, A., Xu, S., Montgomery, M. K., Kostas, S. A., Driver, S. E. and Mello, C. C. (1998) Potent and specific genetic interference by double-stranded RNA in Caenorhabditis elegans. Nature, 391, 806-811.

3. Zamore, P. D. (2002). Ancient pathways programmed by small RNAs. Science, 296, 1265-1269.

4. Ahlquist, A. (2002). RNA-dependent RNA polymerases, viruses, and RNA silencing. Science, 296, 1270-1273.

5. Plasterk, R. H. A. (2002). RNA silencing: the genome's immune system. Science, 296, 1263-1265.

6. Elbashir, S. M., Harborth, J., Lendeckel, W., Yalcin, A., Weber, K. and Tuschl, T. (2001) Duplexes of 21-nucleotide RNAs mediate RNA interference in cultured mammalian cells. Nature, 411, 494-498.

7. Elbashir, S. M., Lendeckel, W. and Tuschl, T. (2001) RNA interference is mediated by 21 and 22 nt RNAs. Genes Dev., 15, 188-200.

8. Caplen, N. J., Parrish, S., Imani, F., Fire, A. and Morgan, R. A. (2001) Specific inhibition of gene expression by small double-stranded RNAs in invertebrate and vertebrate systems. Proc. Natl. Acad. Sci. USA, 98, 9742-9747.

9. Schwarz, D. S., Hutvagner, G., Haley, B. and Zamore, P. D. (2002) Evidence that siRNA function as guides, not primers, in the Drosophila and human RNAi pathways. Molecular Cell, 10, 537-548.

10. Martinez, J., Patkaniowska, A., Urlaub, H., Lührmann, R. and Tuschl, T. (2002) Single-stranded antisense siRNA guide target RNA cleavage in RNAi. Cell, 110, 563-574.

11. Amarzguioui, M., Holen, T., Babaie, E. and Prydz, H. (2003). Tolerance for mutations and chemical modifications in an siRNA. Nucleic Acids Res., 31, 1-7 (in press).

12. Nykänen, A., Haley, B. and Zamore, P. D. (2001) ATP requirements and small interfering RNA structure in the RNA interference pathway. Cell, 107, 309-321.

13. Hammond, S. M., Boettcher, S., Caudy, A. A., Kobayashi, R. and Hannon, G. J. (2001) Argonaute2, a link between genetic and biochemical analyses of RNAi. Science, 293, 1146-1150.

14. Mourelatos, Z., Dostie, J., Paushkin, S., Sharma, A., Charrouz, B., Abel, L., Rappsilber, J., Mann, M. and Dreyfuss, G. (2002). miRNPs: a novel class of ribonucleoproteins containing numerous microRNAs. Genes Dev., 16, 720-728.

15. Hutvagner, G. and Zamore, P. D. (2002) A microRNA in a multiple-turnover RNAi enzyme complex. Science, 297, 2056-2060.

16. Caudy, A. A., Myers, M., Hannon, G. J. and Hammond, S. M. (2002) Fragile X-related protein and VIG associate with the RNA interference machinery. Genes Dev., 19, 2491-2496.

17. Ishizuka, A., Siomi, M. C. and Siomi, H. (2002) A Drosophila fragile X protein interacts with components of RNAi and ribosomal proteins. Genes Dev., 19, 2497-2508.

18. Ambros, V. (2001). MicroRNAs: Tiny regulator with great potential. Cell, 107, 823-826.

19. Holen, T., Amarzguioui, M., Wiiger, M. T., Babaie, E. and Prydz, H. (2002). Positional effects of short interfering RNAs targeting the human coagulation trigger Tissue Factor. Nucleic Acids Res., 30, 1757-1766.

20. Yang, D., Buchholz, F., Huang, Z., Goga, A., Chen, C.-Y., Brodsky, F. M. and Bishop, J. M. (2002) Short RNA duplexes produced by hydrolysis with Escherichia coli RNase III mediate effective RNA interference in mammalian cells. Proc. Natl. Acad. Sci. USA, 99, 9942-9947.

21. Elbashir, S. M, Harborth, J., Weber, K., Tuschl, T. (2002). Methods, 26: 199-213.

22. Harborth, J., Elbashir, S. M., Bechert, K., Tuschl, T., Weber, K. (2001). J Cell Science, 114: 4557-4565.

23. “Antisense 2001: Advances in Epigenetic Medicine, Tokyo, Japan, May 22-23, 2001”. C. A. Stein, Nature Biotechnology, August 2001.

24. O'Neil, N. J., Martin, R. L, Tomlinson, M. L., Jones, M. R., Coulson, A., Kuwabara, P. E., (2001), “RNA-Mediated Interference as a Tool for Identifying Drug Targets”, Am. J. Pharmacogenomics 2001, 1 (1): 45-53.

25. Caplen, N. J., Fleenor, J., Fire, A. & Morgan, R. A. dsRNA-mediated gene silencing in cultured Drosophila cells: a tissue culture model for the analysis of RNA interference. Gene 252, 95-105 (2000).

26. Tuschl T, Zamore P D, Lehmann R, Bartel D P, Sharp P A. Targeted mRNA degradation by double-stranded RNA in vitro. Genes Dev. 13, 3191-3197 (1999).

27. Palauqui J C, Balzergue S. Activation of systemic acquired silencing by localised introduction of DNA. Curr Biol. 9, 59-66 (1999).

28. Elbashir, S. M., Martinez, J., Patkaniowska, A., Lendeckel, W. and Tuschl, T. (2001) Functional anatomy of siRNAs for mediating effiecient RNAi in Drosophila melanogaster embryo lysate. EMBO J., 20:6877-6888

29. Wiiger, M. T., Pringle, K. S. Narahara, N. and Prydz, H. (2000), “Effects of binding of ligand (FVIIa) to induced tissue factor in human endothelial cells.”, Thromb. Res., 98, 311-321.

30. Hvatum, M. and Prydz, H. (1966), “Studies on tissue thromboplastis.”, Biochim. Biophys. Acta, 130, 92-101.

31. Camerer, E., Pringle, S., Skartlien, A. H., Wiiger, M., Prydz, K., Kolsto, A. B. and Prydz, H. (1996), “Opposite sorting of tissue factor in human umbilical vein endothelial cells and Madin-Darby canine kidney epithelial cells”, Blood, 88, 1339-3349.

32. Elbashir, S. M., Martinez, J., Patkaniowska, A., Lendeckel, W. and Tuschl, T. (2001) Functional anatomy of siRNAs for mediating effiecient RNAi in Drosophila melanogaster embryo lysate. EMBO J., 20:6877-6888.

33. Elbashir S M, Harborth J, Lendeckel W, Yalcin A, Weber K, Tuschl T. Duplexes of 21-nucleotide RNAs mediate RNA interference in cultured mammalian cells. Nature 411, 494-498 (2001).

1. A method for utilizing siRNA for the detection of optimal siRNA targeting sites capable of affecting the level of a target nucleic acid, comprising the targeting of one or more sites on an mRNA with one or more siRNA molecules and observing the level of the target nucleic acid.

2. Method according to claim 1, further comprising the following steps:
a) providing a suitable range of siRNA molecules directed towards the target nucleic acid;
b) introducing each of the siRNA molecules into a cell or system containing the target nucleic acid;
c) determining the effect of the siRNA molecules on the level of said target nucleic acid; and
d) identifying the optimal siRNA molecules from the effects determined in (c).

3. Method according to claim 2, wherein the siRNA molecules are provided by chemical synthesis.

4. Method according to claim 2, wherein the siRNA molecules are provided by in vitro transcription.

5. Method according to claim 2, wherein the siRNA molecules are provided by endogenous expression directed by a suitable DNA vector.

6. Method according to claim 2, wherein siRNA molecules are introduced into a suitable cell line.

7. Method according to claim 6,
wherein siRNA molecules are introduced into human keratinocyte cell line HaCaT.

8. Method according to claims 6 or 7, wherein the siRNA molecules are introduces by cationic liposome transfection.

9. Method according to claim 8, wherein the siRNA molecules are introduces by Lipofectamin™2000 transfection.

10. Method according to claims 6 or 7, wherein the siRNA molecules are introduces by electroporation.

11. Method according to claims 6 or 7,
wherein the siRNA molecules are introduces by microinjection.

12. Method according to claims 6 or 7,
wherein the siRNA molecules are introduces by coated gold particles shot into cells (GeneGun in plant cells).

13. Method according to claim 5,
wherein the DNA vector is introduced into an appropriate cell line according to a method selected from the group consisting of the methods according to claims 8-12.

14. Method according to claim 2, wherein the siRNA molecules are introduced into a suitable cell lysate.

15. Method according to claim 2, wherein the siRNA molecules are introduced into a suitable in vitro expression assay.

16. Method according to claim 2, wherein the efficacy of the siRNA molecules are determined by Northern blotting analysis.

17. Method according to claims 2, wherein the efficacy of the siRNA molecules are determined by qRT-PCR.

18. Method according to claim 2, wherein the efficacy of the siRNA molecules are determined by Western analysis.

19. Method according to claim 2,
wherein the efficacy of the siRNA molecules are determined by fluorescent marker.

20. Method according to claim 2, wherein the siRNA efficacy is determined by primer extension analysis.

21. Method according to claims 2, wherein the efficacy of the siRNA molecules are determined by phenotypic marker.

22. Method according to claim 21,
wherein the phenotypic marker indicates one or more specific morphological, proliferative or apoptotic characteristics.


SEQUENCE LISTING (41 sequences)

SEQ ID NO: 1 - DNA, 21 bp, Artificial Sequence
  human siRNA with two “t′s” at 3′ end
gcgcuucaggcacuacaaatt

SEQ ID NO: 2 - DNA, 21 bp, Artificial Sequence
  human siRNA with two “t′s” at 3′ end
gaagcagacguacuuggcatt

SEQ ID NO: 3 - DNA, 21 bp, Artificial Sequence
  human siRNA with two “t′s” at 3′ end
cggacuuuagucagaaggatt

SEQ ID NO: 4 - DNA, 21 bp, Artificial Sequence
  human siRNA with two “t′s” at 3′ end
cccgucaaucaagucuacatt

SEQ ID NO: 5 - DNA, 21 bp, Artificial Sequence
  human siRNA with two “t′s” at 3′ end
uggccggcgcuucaggcactt

SEQ ID NO: 6 - DNA, 21 bp, Artificial Sequence
  human siRNA with two “t′s” at 3′ end
ccggcgcuucaggcacuactt

SEQ ID NO: 7 - DNA, 21 bp, Artificial Sequence
  human siRNA with two “t′s” at 3′ end
cuucaggcacuacaaauactt

SEQ ID NO: 8 - DNA, 21 bp, Artificial Sequence
  human siRNA with two “t′s” at 3′ end
caggcacuacaaauacugutt

SEQ ID NO: 9 - RNA, 21 nt, Artificial Sequence
  siRNA, comprises one base-pairing mutation compared to the wild type siRNA sequence
ccgcuucaggcacuacaaaua

SEQ ID NO: 10 - RNA, 21 nt, Artificial Sequence
  siRNA, comprises one base-pairing mutation compared to the wild type siRNA sequence
gggcuucaggcacuacaaaua

SEQ ID NO: 11 - RNA, 21 nt, Artificial Sequence
  siRNA, comprises one base-pairing mutation compared to the wild type siRNA sequence
gcccuucaggcacuacaaaua

SEQ ID NO: 12 - RNA, 21 nt, Artificial Sequence
  siRNA, comprises one base-pairing mutation compared to the wild type siRNA sequence
gcgguucaggcacuacaaaua

SEQ ID NO: 13 - RNA, 21 nt, Artificial Sequence
  siRNA, comprises one base-pairing mutation compared to the wild type siRNA sequence
gcgcuugaggcacuacaaaua

SEQ ID NO: 14 - RNA, 21 nt, Artificial Sequence
  siRNA, comprises one base-pairing mutation compared to the wild type siRNA sequence
gcgcuucagccacuacaaaua

SEQ ID NO: 15 - RNA, 21 nt, Artificial Sequence
  siRNA, comprises one base-pairing mutation compared to the wild type siRNA sequence
gcgcuucagggacuacaaaua

SEQ ID NO: 16 - RNA, 21 nt, Artificial Sequence
  siRNA, comprises one base-pairing mutation compared to the wild type siRNA sequence
gcgcuucaggcaguacaaaua

SEQ ID NO: 17 - RNA, 21 nt, Artificial Sequence
  siRNA, comprises one base-pairing mutation compared to the wild type siRNA sequence
gcgcuucaggcacuagaaaua

SEQ ID NO: 18 - RNA, 21 nt, Artificial Sequence
  siRNA, comprises two base-pairing mutation compared to the wild type siRNA sequence
gcgcuugagccacuacaaaua

SEQ ID NO: 19 - RNA, 21 nt, Artificial Sequence
  siRNA, comprises two base-pairing mutation compared to the wild type siRNA sequence
gcgcuucagcgacuacaaaua

SEQ ID NO: 20 - RNA, 21 nt, Artificial Sequence
  siRNA, comprises two base-pairing mutation compared to the wild type siRNA sequence
gcgcuucagccaguacaaaua

SEQ ID NO: 21 - RNA, 21 nt, Artificial Sequence
  siRNA, comprises two base-pairing mutation compared to the wild type siRNA sequence
gcgcuucagccacuagaaaua

SEQ ID NO: 22 - RNA, 21 nt, Artificial Sequence
  si RNA, the first two and the last two nucleotides in the 5′-3′ are linked by phosphorothioate bonds, ie. 5′-g*c.
gcgcuucaggcacuacaaaua

SEQ ID NO: 23 - RNA, 21 nt, Artificial Sequence
  si RNA, the last three nucleotides in the 5′-3′ direction are linked by phosphorothioate bonds, ie 5′....-a*u*a-3′
gcgcuucaggcacuacaaaua

SEQ ID NO: 24 - RNA, 21 nt, Artificial Sequence
  si RNA, the first three nucleotides and the last three nucleotides in the 5′-3′ direction are linked by phosphorothioate bonds, ie, 5′-g*c*g....a*u*a-3′
gcgcuucaggcacuacaaaua

SEQ ID NO: 25 - RNA, 21 nt, Artificial Sequence
  si RNA, the first three nucleotides and the last five nucleotides in the 5′-3′-direction are linked by phosphorothioate bonds, ie, 5′-g*c*g.....a*a*a*u*a-3′
gcgcuucaggcacuacaaaua

SEQ ID NO: 26 - RNA, 21 nt, Artificial Sequence
  si RNA, the first and last nucleotides in the 5′-3′ direction are 2-O-methylated.
gcgcuucaggcacuacaaaua

SEQ ID NO: 27 - RNA, 21 nt, Artificial Sequence
  si RNA, the last two nucleotides in the 5′-3′ direction are 2-O-methylated.
gcgcuucaggcacuacaaaua

SEQ ID NO: 28 - RNA, 21 nt, Artificial Sequence
  si RNA, the first, second and last two nucleotides in the 5′-3′ direction are 2-O-methylated.
gcgcuucaggcacuacaaaua

SEQ ID NO: 29 - RNA, 21 nt, Artificial Sequence
  gm cm um  si RNA, the first,  second  and last four nucleotides in the 5′-3′- direction are 2-O-methylated.
gcgcuucaggcacuacaaaua

SEQ ID NO: 30 - RNA, 21 nt, Artificial Sequence
  si RNA, the first and the last nucleotides in the 5′-3′ direction are 2-O-allylated.
gcgcuucaggcacuacaaaua

SEQ ID NO: 31 - RNA, 21 nt, Artificial Sequence
  si RNA, the  last two nucleotides in the 5′ - 3′ direction are 2-O-allylated
gcgcuucaggcacuacaaaua

SEQ ID NO: 32 - RNA, 21 nt, Artificial Sequence
  si RNA, complimenatry strand to SEQ ID22, the first two and the last two nucleotides in the 5′-3′ direction are linked by phosphorothioate bonds, ie. 5′-u*u......c*g-3′
uuuguagugccugaagcgccg

SEQ ID NO: 33 - RNA, 21 nt, Artificial Sequence
  si RNA, complimentary strand to SEQ ID23, the last three nucleotides in the 5′-3′ direction are linked by phosphorothioate bonds, ie 5′....-c*c*g-3′
uuuguagugccugaagcgccg

SEQ ID NO: 34 - RNA, 21 nt, Artificial Sequence
  si RNA,complimentary strand to SEQ ID24, the first three nucleotides and the last three nucleotides in the 5′-3′ direction are linked by phosphorothioate bonds, ie, 5′-u*u*u....c*c*g-3′
uuuguagugccugaagcgccg

SEQ ID NO: 35 - RNA, 21 nt, Artificial Sequence
  si RNA, complimentary strand to SEQ ID25, the first three nucleotides and the last five nucleotides in the 5′-3′ direction are linked by phosphorothioate bonds, ie, 5′-u*u*u.....c*g*c*c*g-3′
uuuguagugccugaagcgccg

SEQ ID NO: 36 - RNA, 21 nt, Artificial Sequence
  si RNA, complimentary strand to SEQ ID26, the first and last nucleotides in the 5′-3′ direction are 2-O-methylated.
uuuguagugccugaagcgccg

SEQ ID NO: 37 - RNA, 21 nt, Artificial Sequence
  si RNA,complimentary strand to SEQ ID27,  the last two nucleotides in the 5′-3′  direction are 2-O-methylated.
uuuguagugccugaagcgccg

SEQ ID NO: 38 - RNA, 21 nt, Artificial Sequence
  si RNA, complimentary strand to SEQ ID 28, the first, second and last two nucleotides in the 5′-3′ direction are 2-O-methylated.
uuuguagugccugaagcgccg

SEQ ID NO: 39 - RNA, 21 nt, Artificial Sequence
  si RNA, complimentary strand to SEQ ID 29, the first,  second and last four nucleotides in the 5′-3′- direction are 2-O-methylated.
uuuguagugccugaagcgccg

SEQ ID NO: 40 - RNA, 21 nt, Artificial Sequence
  siRNA, complimentary strand to SEQ ID30, the first and the last nucleotides in the 5′-3′ direction are 2-O-allylated.
uuuguagugccugaagcgccg

SEQ ID NO: 41 - RNA, 21 nt, Artificial Sequence
  siRNA, complimentary strand to SEQ ID31, the last two nucleotides in the 5′-3′ direction are 2-O-allylated
uuuguagugccugaagcgccg